(19) United States
(12) Patent Application Publication
(10) Pub. No.: US 2023/0390110 A1
(43) Pub. Date: Dec. 7, 2023

(54) SMART IMPLANTABLE MICRO DEVICE FOR THERAPEUTIC ACTIVE FLOW CONTROL AND ASSOCIATED SYSTEMS AND METHODS FOR USE

(71) Applicant: Injectsense, Inc., Emeryville, CA (US)

(72) Inventors: Jose Padovani, San Jose, CA (US); Hakan Ates Gurcan, Los Gatos, CA (US); Ariel Cao, Oakland, CA (US); David Huber, Palo Alto, CA (US)

(21) Appl. No.: 18/134,988

(22) Filed: Apr. 14, 2023

Related U.S. Application Data
(60) Provisional application No. 63/331,202, filed on Apr. 14, 2022.

(51) Int. Cl. A61F 9/00; A61B 3/16; A61F 9/007; A61N 1/36

(52) U.S. Cl. CPC A61F 9/0017; A61N 1/36046; A61F 9/00781; A61B 3/16

(57) ABSTRACT

The disclosed apparatus, systems and methods relate to a smart minimally invasive glaucoma surgery (Smart-MIGS) device configured for passive and active flow. Various implementations are defined by a combination device that integrates intraocular pressure (IOP) monitoring, drug delivery and a drainage function for pressure control in the body or bodily region of a subject.

### CROSS-REFERENCE TO RELATED APPLICATIONS

This application claims priority to U.S. Provisional Application No. 63/331,202 filed Apr. 14, 2022 entitled “Smart Micro-Shunt for Therapeutic Intraocular Pressure Control and Associated Apparatus, Systems and Methods for Use,” which is hereby incorporated by reference in its entirety under 35 U.S.C. § 119(e).

### TECHNICAL FIELD

The disclosed technology relates generally to a smart minimally invasive glaucoma surgery (Smart-MIGS) device. Various implementations are defined by a combination device that integrates intraocular pressure (IOP) monitoring, and a drainage function for pressure control and/or as a pumping function for pressure control by draining fluid volume from the eye or delivering pressure-modulating drugs. The disclosed device, systems and methods use a 3D integrated layer stack that allows different incremental options up to an overall system that automatically regulates IOP. The device allows passive or active aqueous humor drainage, as dictated by the IOP monitoring, to shape the IOP waveform and control IOP over time.

### BACKGROUND

For many years, the global glaucoma market was relatively inactive, with minimal innovation on both the pharmaceutical and device side. No significant new classes of pharmaceutical agents have been approved since the first prostaglandin (latanoprost) gained clearance in 1996. The device side has languished with older laser technologies (trabeculoplasty) and relatively ineffective and/or risky surgical procedures (trabeculectomy and shunts) being the mainstays in treating drug-resistant or non-compliant patients.

In MIGS-based glaucoma therapy, the prior art is limited to passive mechanical drainage devices. These devices are typically focused on increased drainage or outflow. Typically, these MIGS devices only provide a fixed hydraulic resistance, and, in some cases, it could require more than one device to achieve the targeted IOP value by the therapy. The main disadvantage of this approach is that this device only offers an open loop model with very limited or no flexibility in terms of incremental IOP lowering effect. The other issue is that lack of true IOP continuous data to assess how the aqueous outflow varies and true impact on IOP as a dynamic state. The endpoint of reducing the IOP with a MIGS device is still plagued with unknown parameters within circadian cycle and if the therapy at peak IOP (at night) is adequate to stop the progression of the disease.

The static drainage of these MIGS devices of the prior art, regardless of the discharge pathway, is not aligned with the dynamic state of the inflow-outflow and related variability IOP (linked to body position and perfusion pressure).

Thus, there is a need in the art for dynamic solutions, and in particular, devices, systems and methods for the controlled passive or active drainage of the eye for treating elevated intra-ocular pressure disorders such as glaucoma.

Further, there is a need in the art for dynamic solutions for treating elevated intra-ocular pressure disorders such as glaucoma, and in particular, devices systems and methods for the controlled drainage of fluid from—and delivery of pharmaceuticals to—the eye.

### BRIEF SUMMARY

Discussed herein are various devices, systems and methods relating to an implantable active micro-pump device for use in the sensing of fluid pressure and actuation of fluid flow. While the disclosed implementations focus on intraocular pressure, certain implementations can be used for pressure control in other areas of the body, as well as in the localized drug delivery from a drug reservoir for cancer treatment, pain management and the like, as would be readily appreciated.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One Example includes an implantable active micro-shunt. Other embodiments of this aspect include corresponding computer systems, apparatus, and computer programs recorded on one or more computer storage devices, each configured to perform the actions of the methods.

In Example 1, an implantable active micro-shunt, comprising an actuation and sensing module, wherein the actuation and sensing module is configured to detect intraocular pressure (IOP) and actuate flow to a suprachoroidal space of a subject.

In Example 2, the implantable active micro-shunt of Example 1, further comprising a flexible housing.

In Example 3, the implantable active micro-shunt of any of Examples 1-2, wherein the actuation and sensing module comprises a plurality of laminar layers.

In Example 4, the implantable active micro-shunt of any of Examples 1-3, wherein the plurality of laminar layers comprises a microelectromechanical systems (MEMS).

In Example 5, the implantable active micro-shunt of any of Examples 1-4, wherein the plurality of laminar layers comprises an energy storage component.

In Example 6, the implantable active micro-shunt of any of Examples 1-5, wherein the plurality of laminar layers comprises an active drainage layer comprising at least one electrode.

In Example 7, the implantable active micro-shunt of any of Examples 1-6, wherein the plurality of laminar layers comprises an ASIC.

In Example 8, an implantable active micro-pump device, comprising an active channel portion, and an actuation and sensing module, comprising: a pressure sensor configured to detect IOP, a stored energy component, and an application specific integrated circuit (ASIC) in operational communication with the active channel portion, stored energy component and pressure sensor, wherein the ASIC is configured to actuate electrohydrodynamic actuation of fluid in the active channel portion in response to detected IOP via the stored energy component.

In Example 9, the implantable micro-pump device of any of Examples 1-8, further comprising a MEMS in operational communication with the ASIC and active channel portion.

In Example 10, the implantable micro-pump device of any of Examples 1-9, further comprising one or more electrodes disposed in the active channel configured for electrohydrodynamic actuation.

In Example 11, the implantable micro-pump device of any of Examples 1-10, wherein the pressure sensor is a capacitive pressure sensor comprising a membrane.

In Example 12, the implantable micro-pump device of any of Examples 1-11, wherein the one or more electrodes are configured to actuate electrohydrodynamic fluid flow in response to applied voltage.

In Example 13, the implantable micro-pump device of any of Examples 1-12, wherein the ASIC is in electrical communication with the one or more electrodes.

In Example 14, the implantable micro-pump device of any of Examples 1-13, further comprising a power and telemetry coil in operational communication with the ASIC.

In Example 15, an implantable active micro-pump device, comprising: an active channel portion, and an actuation and sensing module, comprising: a MEMS comprising a pressure sensor configured to detect IOP, a stored energy component, and an application specific integrated circuit (ASIC), wherein the ASIC is in operational communication with the active channel portion, MEMS, stored energy component and pressure sensor and is configured to actuate electrohydrodynamic actuation of fluid in the active channel portion in response to detected IOP via the stored energy component.

In Example 16, an implantable active micro-pump device, comprising: an active channel portion, and an actuation and sensing module, comprising: a MEMS comprising a pressure sensor comprising a membrane and configured to detect IOP, at least one electrode, and an ASIC, wherein the actuation and sensing module is configured to actuate electrohydrodynamic actuation of fluid in the active channel portion over time in response to detected IOP via the stored energy component via application of variable voltage.

In Example 17, an actuation and sensing module, comprising: a MEMS comprising a pressure sensor configured to detect IOP, and an ASIC, wherein the actuation and sensing module is configured to actuate electrohydrodynamic actuation of fluid in an active channel portion over time in response to detected IOP.

In Example 18, a method of regulating IOP, comprising: implanting an actuation and sensing module in the suprachoroidal space, the actuation and sensing module comprising: a MEMS comprising a pressure sensor configured to detect IOP, and an ASIC, sensing IOP via the pressure sensor, and actuating electrohydrodynamic fluid flow in response to sensed IOP.

In Example 19, an implantable active micro-shunt, comprising: a housing, an actuation and sensing module, comprising: a MEMS comprising a pressure sensor configured to detect IOP, a stored energy component, a power and telemetry coil, and an application specific integrated circuit (ASIC), wherein: the actuation and sensing module is configured to actuate electrohydrodynamic actuation of fluid in an active channel portion over time in response to detected IOP, and the power and telemetry coil is configured to communicate with a reader and charge the stored energy component.

In some Examples, the implantable shunt device comprises an integrated circuit (IC) and a MEMs device. The MEMs device can include a pressure sensing device, such as an IOP sensor.

In some Examples, the sensor is a pressure sensor configured for measurement of intraocular pressure (IOP) and the device is configured for implantation in an eye of a patient.

In some Examples, the implantable shunt device is configured for on-demand or autonomous operation.

In some Examples, the implantable shunt device is configured to regulate IOP in real-time.

Other embodiments of these Examples include corresponding computer systems, apparatus, and computer programs recorded on one or more computer storage devices, each configured to perform the actions of the methods.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1A depicts a perspective view of the implantable device and subcomponents, according to one implementation.

FIG. 1B depicts several views of the housing, according to certain implementations.

FIG. 1C depicts a flow diagram of the system, according to one implementation.

FIG. 2A is a schematic of an eye with the implantable device implanted in the suprachoroidal space, according to certain implementation.

FIG. 2B is a further schematic of an eye with the implantable device implanted in the suprachoroidal space showing the unconventional pathway, according to certain implementations.

FIG. 3 is a perspective view of the implantable device showing the coil, according to one implementation.

FIG. 4 is a perspective view of the implantable device showing the sensing and actuation module components, according to one implementation.

FIG. 5 is a cross-sectional view of the implantable device showing the sensing and actuation module components, according to one implementation.

FIG. 6 is a system architecture diagram of the implantable device showing the relationship of various sensing and actuation module components, according to one implementation.

FIG. 7 is a sectional view of the implantable device channel showing the electrode and microchannels, according to one implementation.

FIG. 8 is a sectional view of the implantable device channel showing the electrode and microchannels, according to another implementation.

FIG. 9 contains cross-sectional views of the module showing various channel implementations.

FIG. 10A is a flow chart showing the device in use, according to one implementation.

FIG. 10AB is a flow chart showing the device in use, according to another implementation.

### DETAILED DESCRIPTION

The various embodiments disclosed or contemplated herein relate to an implanted smart micro-shunt (SMS) or smart micro-pump (SMP) device and associated systems and methods designed to address IOP by controlled drainage of the eye or dynamic delivery of pressure modulating drugs. These implementations include closed-loop integrated sensing and actuation devices, systems and methods for use in treating elevated intra-ocular pressure disorders, such as glaucoma and others understood by those of skill in the art. Certain implementations implement on-demand or automated flow actuation which achieve higher flow rates than passive MIGS devices and can improve IOP consistency over time.

It is understood that while certain implementations of the device are designed to move fluid in a passive way, such as via shunting, while an aspect of further implementations is active fluid transfer, and in these implementations the device 10 utilize electrokinetic or electrohydrodynamic technologies to facilitate the flow of fluid through the device, such as via electrokinetic mechanisms such as electroosmosis pumping or the other approaches described herein and understood by those of skill in the art. It is therefore understood that in certain implementations, for example, the device is an SMP device for use in lop pressure regulation or drug delivery implementations, such that the use of the terms “shunt” or “pump” for example should not be limiting to any individual implementation, nor should the use of the terms “electrokinetic” or “electrohydrodynamic” be understood to be exclusive or limiting as to any individual implementation.

Further, various of the approaches and technologies discussed herein can be combined with any of the teachings of any of U.S. Ser. No. 17/199,858 entitled “Hermetically Sealed Implant Sensors With Vertical Stacking Architecture” filed on Mar. 12, 2021, U.S. Ser. No. 17/550,160 entitled “Methods and Devices for Implantation of Intraocular Pressure Sensors” filed Dec. 14, 2021, U.S. Ser. No. 14/789,942 entitled “Ultra low power charging implant sensors with wireless interface for patient monitoring” filed Jul. 1, 2015, U.S. Ser. No. 17/566,930 entitled “Hermetic Heterogeneous Integration Platform for Active and Passive Electronic Components” filed Dec. 31, 2021, and U.S. Ser. No. 17/688,408 entitled “Circadian Intraocular Pressure Profile and Methods of Treatment” filed Mar. 7, 2022, each of which is incorporated by reference in its entirety.

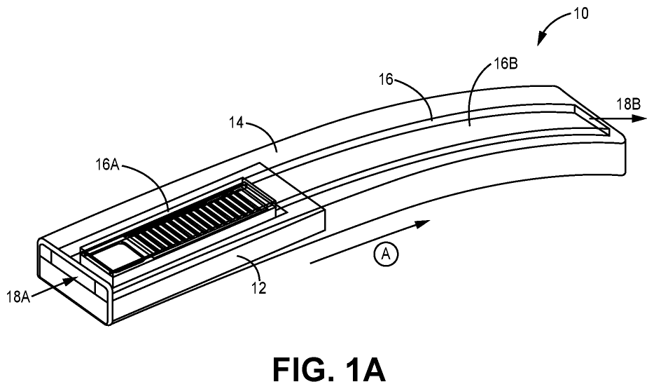

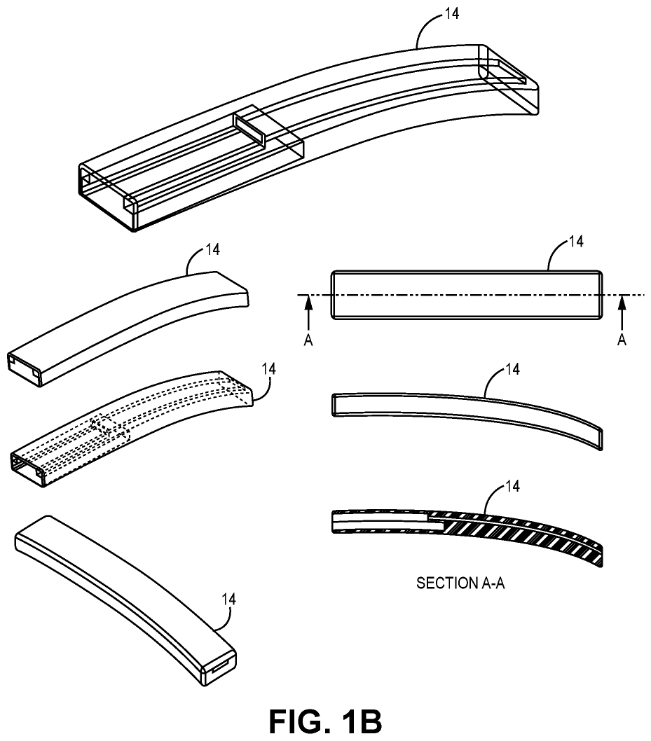

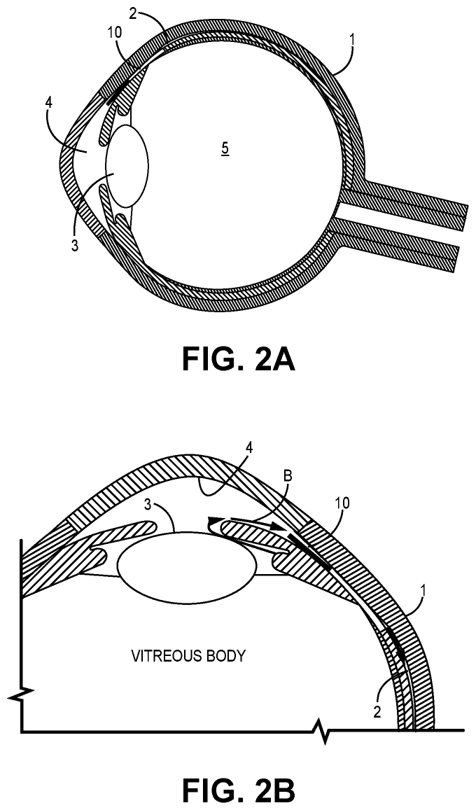

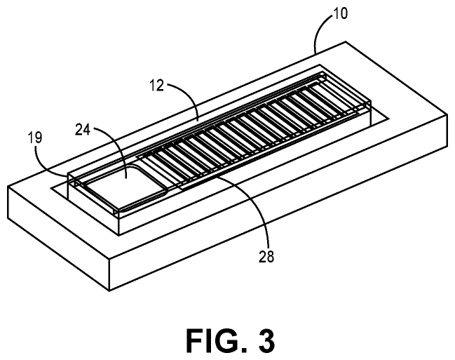

Turning to the drawings in greater detail, FIG. 1A depicts one implementation of the implantable device 10. In various implementations, the implantable device 10 is an implantable SMS or SMP device sized to be implantable in a region of interest, such as the eye, as discussed in relation to FIGS. 2-3. In the implementations of FIGS. 2-3, the implantable SMS device is configured to drain the aqueous humor (fluid) from the eye. In certain active implementations, the implanted device 10 senses the intraocular pressure (IOP) and is configured to actively promote the flow of fluid in response to the sensed IOP so as to regulate the IOP, as would be readily understood. In various implementations, the SMS device 10 disclosed herein is an on-demand implantable device 10 that is capable of reducing IOP in response to external commands, while in further implementations the device 10 is autonomous, in that it is actuated in direct response to sensed pressure through the variable application of voltage/amperage, as described in further detail herein.

The challenge of the active drainage integration is located within both form factor and power. Electrohydrodynamic pumping provides a form factor already proven in microfluidic channels. Various implementations dynamically adjust the outflow through electrohydrodymanics to regulate, adjust or otherwise cap lop to a target value.

In certain aspects, the dynamic IOP value is averaged to remove the fluctuation due to ocular pulse amplitude (OPA). This average value can be also tracked in terms of a trend or first-order derivative in order to potentially increase the micropump actuation rate. Further, it is appreciated that the flow rate can be regulated over time to account for rhythmic fluctuations in IOP and to smooth or otherwise shape the curve so as to maintain relatively consistent IOP over time within the bounds of normal biological fluctuations and independent of subject orientation or activity. In various implementations, the data shaping parameters can be unique to each subject, with parameters controlled by a clinician and programmed to the SMS, as would be appreciated.

FIG. 1B depicts several views of the housing 14. In certain implementations, the flexible housing 14 is able to accomplish several serves multiple purposes. In addition to defining an opening for the module 12 in certain implementations it is configured for passive drainage that delivers fluid to the suprachoroidal space. The module 12 can be located in multiple positions within the housing 14 such as proximal, distal or fully embedded. Passive drainage can be implemented as a single or multiple lumen channel 16 and with additional porous surface for promoted diffusion outflow. Both channel walls and channel lumen can be made of porous material, as would be understood.

Returning to the implementation of FIG. 1A, the implantable device 10 has an actuation and sensing module 12 disposed within a housing 14 defining a channel 16 therethrough with an inflow opening 18A and outflow opening 18B. In various implementations, the cross-sectional channel 16 dimensions and length can be variable and optimized to specific applications and can optionally utilize porous and/or multi-channel outflow configurations, as discussed further below.

As shown in FIGS. 2A-2B, the implantable device 10 according to certain implementations is configured to be implanted by a minimally invasive procedure on the suprachoroidal space 2 of the eye 1 to enhance, on demand or autonomously, the anterior chamber 4 drainage away from the lens 4 and vitreous body 5 through the suprachoroidal space (also referred to is the “unconventional pathway”) and into the venous blood stream, as is shown in FIG. 2B at reference arrow A. To implant the SMS device 10 of these implementations, a surgical insertion such as an ab interno procedure can be performed, as would be readily understood by those of skill in the art. It is understood that in various alternate implementations, the device 10 according to certain implementations can be an SMP device 10 that can be implanted at other locations in the body to control the flow of fluids, such as to control drainage or pressure of other fluids throughout the body, or for use in drug delivery applications, such as in the brain or elsewhere.

The anterior chamber 4 is the focus of IOP monitoring and also adjusted based on the supplemental outflow which needs to be applied. In certain of the active drainage implementations discussed herein, the reduction of IOP will be needed only when the inflow-outflow is in unbalanced state. The reduction in pressure can be calculated by the continuous volume to be extracted from the anterior chamber 4 depending on the active drainage operating parameters.

As shown in the implementations of FIGS. 1A-2B and elsewhere, in various implementations the housing 14 is curved and/or flexible for ease of implantation and adjustment as well as comfort. That is, the housing 14 and device 10 can be curved, so as to more appropriately be contained within the eye 1, as would be appreciated by those of skill in the art. It is of course possible that in alternative implementations, other possible drainage locations in the eye can also be used in conjunction with the SMS device 10 with different housing 14 and sensing and actuation module 12 geometries and configurations. In certain implementations, the housing 14 is a molded polymer or other appropriate material that would be understood by those of skill in the art. Further views of the housing 14 according to certain implementations are shown in FIG. 1B.

Returning to the implementation of FIG. 1A, it is appreciated that the channel 16 and openings 18A, 18B are sized and otherwise configured to allow for the passage of aqueous humor through the channel 16 from the inflow opening 18A to the outflow opening 18B, thus passing through the device 10 and module 12, as shown at reference arrow A. It is further understood that the channel 16 according to certain implementations comprises an active portion 16A that is in direct fluidic communication with the module 12, and one or more passive portions 16B that is/are either downstream (as shown) and/or upstream (not shown). That is, fluid flow in the active portion 16A is actuated by the module 12 and fluid flow in the passive portion 16B is passive other than as affected by the active flow, as would be understood.

In implementations like those of FIG. 1A, the device 10 achieves active drainage of the eye by pumping aqueous humor (fluid) via the sensing and actuation module 12 in response to measured IOP, as is described further herein. In various implementations, dynamically controlled IOP is achieved in certain implementations by including the active drainage channel portion 16A within the device. In these implementations, the active drainage channel 16A provides a supplemental outflow to complement the natural outflow and/or the supplemental outflow of a passive MIGS device. Active drainage frequency and duration are determined by the closed feedback loop IOP pressure sensor 24 described below. The resulting shape of the IOP waveform can be controlled by the active feedback system and tuned to stay within clinically established thresholds or otherwise most closely achieve a target flow rate. Active drainage has the advantage over purely passive systems that it does not rely on the naturally fluctuating differential pressure between spaces in the eye and can be especially effective in increasing outflow during naturally low-flow conditions (such as night), where it can smooth the curve. Those of skill in the art will appreciate that in the on demand and autonomous implementations of the active device/SMP discussed herein, the active channel portion 16A operates as a passive channel when the device 10 is not being actuated due to understood fluid dynamics/pressure differentials, but can be actuated to promote flow in the form of a smart micro-pump via, for example, electrothermal or electroosmosis pumping or the other approaches described herein.

FIG. 3 depicts the sensing and actuation module 12 with a power and telemetry coil 19 for external communications, as would be understood. In various implementations the coil 19 is a gold wire loop embedded in a biocompatible material such as ceramic, glass, polymer or other suitable material. In these implementations, the sensing and actuation module 12 is a 3D integrated device in direct communication with the power and telemetry coil 19 and including a variety of optional subcomponents.

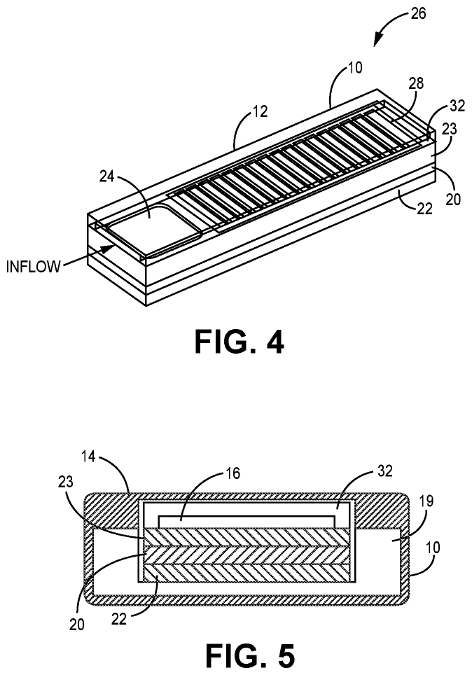

In certain implementations, like those of FIGS. 4-6, the sensing and actuation module 12 comprises a variety of components and sub-components. As shown in FIGS. 4-5, in various implementations, the sensing and actuation module 12 has a laminar structure with a variety of the components disposed throughout, as would be appreciated by those of skill in the art. In various implementations, the sensing and actuation module 12 comprises a laminar silicon wafer bonded to create a monolithic solid state integrated circuit.

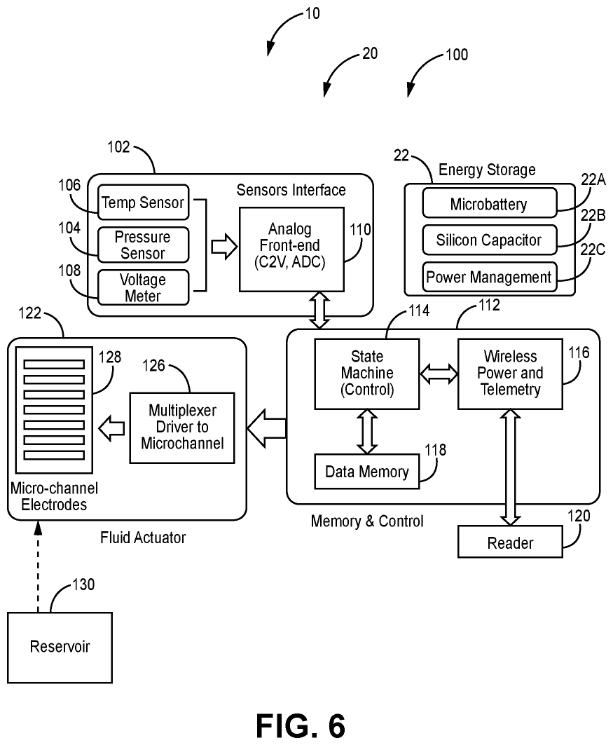

In certain implementations, the sensing and actuation module 12 has an optional application-specific integrated circuit (ASIC) 20 for signal processing and control, as would be readily understood and is explained in further detail below in reference to FIG. 6. As described herein, in various implementations, the ASIC is operationally integrated with the various components of the module 12 and is used to perform functions such as fluid actuation, active channel 16A control function, power management, input and output of data, data telemetry and command of frequency and voltage to perform actuation via electrodes 28, controlling the voltages applied at determined amplitudes, as well as analog to digital conversion from a capacitive pressure sensor 24 to the ASIC 20. The active channel 16A These implementations of the device 10 also comprise an optional energy storage component 22 also discussed further in relation to FIG. 6.

Continuing with the implementations of FIGS. 4-5, certain implementations of the device comprise a micro-electromechanical systems (MEMS) layer 23 that is operationally integrated with the ASIC 20 as well as the active channel portion 16A. In various implementations, the MEMS 23 comprises an optional pressure sensor 24. In the implementation shown in FIGS. 1 and 3-5, the pressure sensor 24 is disposed distally, that is adjacent to the inflow opening 18A, though it is readily appreciated that the pressure sensor 24 can be disposed in alternate locations in further implementations. In various implementations, the pressure sensor 24 comprises a membrane and is a capacitive pressure sensor 24 that is operationally integrated with the ASIC 20 for control of actuation, as described herein.

Various implementations of the module 12 comprise an electrohydrodynamic layer (shown generally at 26) disposed within the active channel portion 16A with one or more electrodes 28 disposed in parallel thereon and, optionally, one or more electrode channel 30 defined therethrough. In response to variable voltage/amplitude commands, the electrodes 28 are able to actuate fluid flow through the channel 16 so as to promote a decrease in IOP or other objective as would be readily apparent. In various implementations, the electrodes 28 and electrode channels 30 are built directly on the MEMS 23, while in alternate implementations they can be disposed on the housing 14/drainage layer 32, as would be readily understood. Further, in various implementations, the electrodes 28/channels 30 are disposed perpendicular to or parallel to the direction of flow in the channel 16, as well as in angled or more complex patterns. Further, various implementations have electrodes 28 that are positioned at various heights, for example by having steps up and down along the channel 16 or by depositing taller and shallower electrodes 28 within the channel 16, as would be understood.

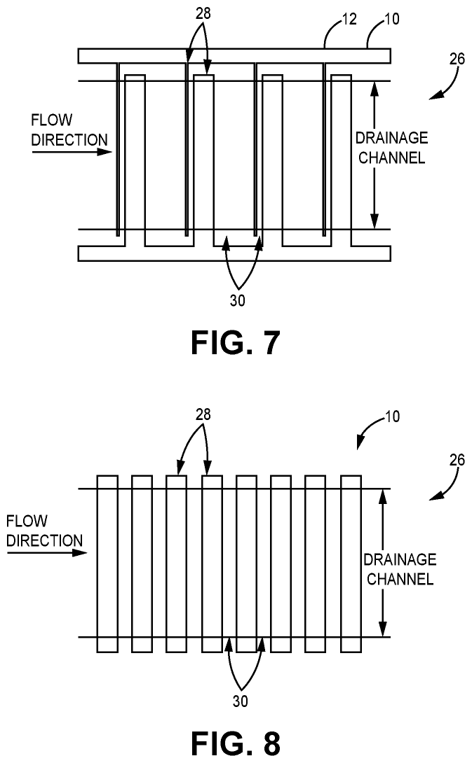

In various implementations, the electrode 28 can comprise gold, titanium or other material. In certain implementations, the electrode geometry 28 can be a variety of shapes, such as planar, 3D, and have a variety of arrangements, such as the symmetric and asymmetric configurations shown in FIGS. 7-8, featuring a range of possible dimensions. Further, in certain implementations the electrode 28 or electrodes 28 are coated with an appropriate dielectric material, such as SiO2, ALD alumina or others as would be understood.

In use according to these implementations, active pumping is accomplished by the use of electrohydrodynamics or electrokinetics (EK), where the application of an electric field induces a force on liquid or fluid (such as aqueous humor) with a net electrical charge (i.e., positive or negative) that causes flow without any mechanically moving parts. Electrohydrodymanics/EK encompasses a variety of pumping mechanisms, such as induction, electrothermal pumping such as AC electrothermal pumping (ACET), electroosmosis (EO) such as DC electroosmosis pumping, alternating current electroosmosis (ACEO), and induced charge electroosmosis (ICEO).

For example, certain non-limiting approaches to promoting flow are described here. Regions of net charge can be created in the fluid via charged surfaces (DC electrosmotic pumping), electrodes within the channel (ICEO and ACEO), charge separation caused by electric fields interacting with conductivity gradients in the fluid (ACET), or other approaches understood in the art. It is understood that in certain implementations, applied electric fields exert a net force on those regions causing fluid motion, and fluid motion in those regions is transmitted to adjacent neutral fluid flow via viscosity, leading to bulk flow. Further mechanisms would be readily apparent to those of skill in the art.

As would be appreciated, in the implementations of FIGS. 7-8, electric fields are generated with electrodes 28, which may be fabricated on the top or bottom surface of a microchannel 23 and connected to the ASIC 20 for control.

As shown in FIG. 4, the sensing and actuation module 12 within the housing 14 further defines a drainage layer 32 along the length of the housing that defines the uppermost aspect of the channel. In various implementations, the drainage layer 32 has a glass or silicon layer or cap that defines the channel 16 opposite the MEMS 23. Various implementations of the channel drainage layer 32/channel 16 can comprise a surface coating layer material of a defined thickness to facilitate flow, as well as optionally comprise a porous material at the inlet 18A, outlet 18B or both.

FIG. 5 depicts a cross section of the sensing and actuation module 12 according to certain implementations, depicting the individual layers of the laminar structure, the channel defined therethrough and the surrounding housing 14.

Returning to the ASIC 20 of FIGS. 4-5, in various implementations, the ASIC 20 is structured and arranged to perform a plurality of functions, as shown for example in the diagram of FIG. 6 generally at 100. That is, in various implementations the ASIC 20 is configured to interface with sensors (box 102), such as one or more of a pressure sensor (box 104, also shown in FIGS. 4-5 and elsewhere at 24), a temperature sensor (box 106), and/or a voltage meter (box 108). In various implementations, the ASIC 20 is configured to receive analog sensor input for a digital conversion (box 110), such as via capacitance-to-voltage (C2V) analog digital converter (ADC) or similar technologies.

In various implementations, these various sensor signals (box 102) are converted to digital signals and are in electronic communication with the memory and control components (box 112). Such memory and control components (box 112) can include one or more components for performing state control (box 114), wireless power and telemetry (box 116) interfaces configured to perform data input/output functions with optional internal memory (box 118) or external devices (box 120). In exemplary implementations, external devices (box 120) such as readers can be utilized to receive data from the memory and control components (box 112) for use in external analysis and the like, as would be appreciated. Such readers/external devices (box 120) can further provide recharging and other power-related functions. It is understood that the external devices (box 120) are in two-way wireless communication with the device via the coil (shown in FIG. 3 at 19) via radiofrequency (RF), near-field communication (NFC), radiofrequency identification (RFID), optical/light-based (including IR and near-IR) transfer or other similar technologies understood in the art for the transfer of data, energy or other signals wirelessly.

As is also shown in the architecture of FIG. 6, various implementations of the closed-loop sensing and actuation module's 12 ASIC 20 comprise an actuation interface or actuator (box 122) that can be configured to receive output flow commands such as voltage and amperage commands from the memory and control components (box 112 via arrow 124) to actuate fluid flow. An optional driver (box 126) such as a multiplexer driver can be provided to control the commands issued to the micro-channel electrodes (box 128, shown elsewhere at 28). It is readily appreciated that in implementations that include drug delivery, a reservoir (box 130) is in fluidic communication with the fluid actuator (box 122) or pump, as would be readily understood. In these implementations, the pump (box 122) is configured to draw from the reservoir (box 130) on command to deliver the relevant drug to the subject as desired.

A further optional component shown in the implementations of FIGS. 4-5 and 6 is an optional stored energy component 22. In various implementations, the stored energy component 22 comprises one or more of a microbattery 22A, capacitor 22B and/or other energy source or power management component 22C that is configured for energy storage and on demand use to power the various components of the device 10 and ASIC 20. In certain representative examples, an optional rechargeable microbattery 22A is utilized for autonomous operation. In certain implementations, the rechargeable microbattery is a Lithium ion or Lithium polymer battery or other similar form of rechargeable battery 22A.

In various implementations, the energy component 22 is charged or recharged wirelessly, such as via an external charging unit (not shown) utilizing radiofrequency uncoupled charging technologies. In various implementations, the energy component 22 can be charged periodically, such as while the subject is sleeping, via a patch or other article fitted outside the eye of the subject, as has been previously described. Power consumption is mitigated by tuning the pumping rate of the active drainage to enable pressure reduction over a period of time.

Returning to the fluidic actuation of the module 12, in various implementations the type of electrohydrodynamic physics used determine the range of actuation voltages, frequencies and, ultimately, the flow rate that can be achieved. Similarly, the types of electrodes 28 and dielectrics used can vary with each approach and can be configured with different 2D geometries, materials, thicknesses and arrangements, as would be readily appreciated by those of skill in the art. For example, FIG. 7 shows a planar arrangement of electrodes 28 with asymmetric width at the bottom of a microchannels 30, such as would be used in conjunction with ACEO implementations.

FIG. 8 shows an alternate configuration with electrodes 28 of symmetric width. The width and spacings between electrodes 28 and microchannel 30 geometries are used as variables to optimize the resulting pumping flow rate for a given voltage, channel and liquid type, as would be appreciated. It is further appreciated that 3D electrode 28 arrangements can also be used in combination with the configuration options presented above to increase the resulting flow rate.

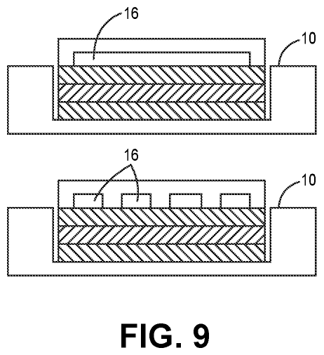

It is further understood that channel design can also be used to optimize pumping efficiency. Additionally, as shown in the implementations of FIG. 9, single or multiple parallel channels 16 can be fabricated with integrated electrodes disposed therein. Channel 16 width and height are also variables in the flow optimization process, as would be readily appreciated by the skilled artisan.

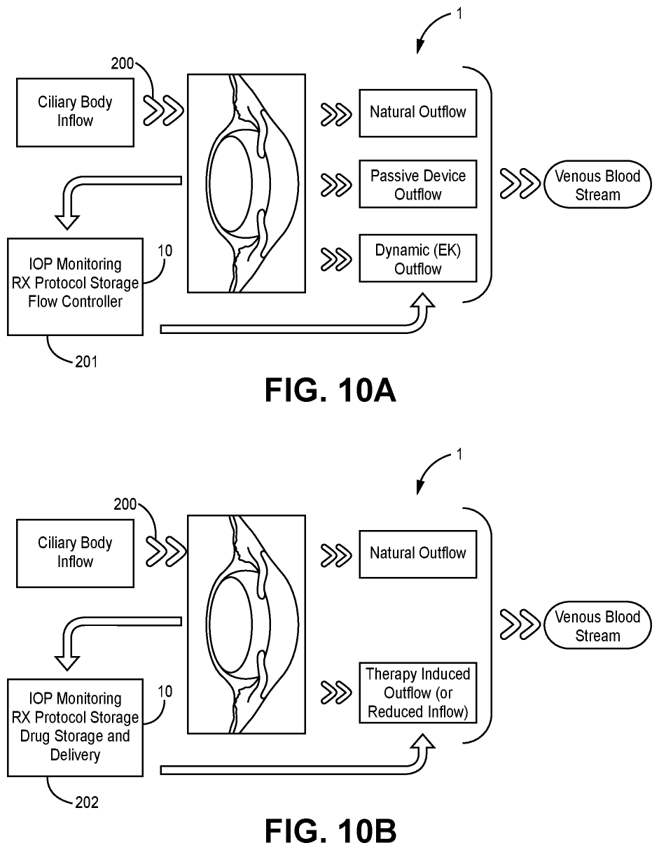

FIG. 10A depicts another flow chart system 1 implementation demonstrating the use of the implantable device 10 used in an adaptive drainage application. FIG. 10B depicts another flow chart system 1 implementation demonstrating the use of the implantable device 10 in a drug delivery implementation. In these implementations, there is inflow (box 200) into the subject body, such as the eye. Monitoring, such as IOP monitoring, is performed by the device, as is optional program or protocol execution, such as flow control 201 execution and/or drug delivery 202 execution, to promote or regulate flow through the device 10.

In various implementations, the device 10 is configured to regulate or induce fluid flow into (or out of?) the body as determined by detected or programmed thresholds or other parameters. For example, in certain implementations, drug delivery can be performed on the basis of a time course or in response to sensed conditions in the body or other factors as would be readily appreciated.

It is further appreciated that any of the above concepts can be used as described above, individually or in combination, or can be modified according to various alternatives. Such alternatives include use of different dielectric materials and/or fabrication methods (e.g. multilayer thick film process, low-temperature co-fired ceramic (LTCC), 3D printing/scaffolding, silicon). In some embodiments, the design could include a single cavity or could include multiple cavities for both active and passive electronic components.

It is appreciated that electrokinetic or electrohydrodynamic flow offers certain advantages, certain non-limiting examples being that the fluid is driven simply by application of an electric potential, thus there are no mechanical components to break down or cause vibration; that these implementations are compatible with other electronics platforms, are well established for in-vitro (lab-on-a-chip) applications, and the different modalities provide options to address risk.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed systems and methods, the subject has been diagnosed with a need for treatment.

Although the disclosure has been described with reference to preferred embodiments, persons skilled in the art will recognize that changes may be made in form and detail without departing from the spirit and scope of the disclosed apparatus, systems and methods.

1. An implantable active micro-shunt, comprising an actuation and sensing module, wherein the actuation and sensing module is configured to detect intraocular pressure (IOP) and actuate flow to a suprachoroidal space of a subject.

2. The implantable active micro-shunt of claim 1, further comprising a flexible housing.

3. The implantable active micro-shunt of claim 1, wherein the actuation and sensing module comprises a plurality of laminar layers.

4. The implantable active micro-shunt of claim 3, wherein the plurality of laminar layers comprises a microelectromechanical systems (MEMS).

5. The implantable active micro-shunt of claim 3, wherein the plurality of laminar layers comprises an energy storage component.

6. The implantable active micro-shunt of claim 3, wherein the plurality of laminar layers comprises an active drainage layer comprising at least one electrode.

7. The implantable active micro-shunt of claim 3, wherein the plurality of laminar layers comprises an application specific integrated circuit (ASIC).

8. An implantable active micro-pump device, comprising:
a) an active channel portion; and
b) an actuation and sensing module, comprising:
i) a pressure sensor configured to detect intraocular pressure (IOP);
ii) a stored energy component; and
iii) an application specific integrated circuit (ASIC) in operational communication with the active channel portion, stored energy component and pressure sensor,
wherein the ASIC is configured to actuate electrohydrodynamic actuation of fluid in the active channel portion in response to detected IOP via the stored energy component.

9. The implantable active micro-pump device of claim 8, further comprising a microelectromechanical systems (MEMS) in operational communication with the ASIC and active channel portion.

10. The implantable active micro-pump device of claim 9, further comprising one or more electrodes disposed in the active channel configured for electrohydrodynamic actuation.

11. The implantable active micro-pump device of claim 10, wherein the pressure sensor is a capacitive pressure sensor comprising a membrane.

12. The implantable active micro-pump device of claim 10, wherein the one or more electrodes are configured to actuate electrohydrodynamic fluid flow in response to applied voltage.

13. The implantable active micro-pump device of claim 10, wherein the ASIC is in electrical communication with the one or more electrodes.

14. The implantable active micro-pump device of claim 10, further comprising a power and telemetry coil in operational communication with the ASIC.

15. An implantable active device, comprising:
a) an active channel portion; and
b) an actuation and sensing module, comprising:
i) a microelectromechanical systems (MEMS) comprising a pressure sensor configured to detect intraocular pressure (IOP);
ii) a stored energy component; and
iii) an application specific integrated circuit (ASIC),
wherein the ASIC is in operational communication with the active channel portion, MEMS, stored energy component and pressure sensor and is configured to actuate electrohydrodynamic actuation of fluid in the active channel portion in response to detected TOP via the stored energy component.

16-19. (canceled)

20. The implantable active device of claim 15, wherein the pressure sensor comprises a membrane.

21. The implantable active device of claim 15, wherein the actuation and sensing module is configured to actuate electrohydrodynamic actuation of fluid in the active channel portion via application of variable voltage.

22. The implantable active device of claim 15, wherein the actuation and sensing module configured for implantation into the suprachoroidal space.

23. The implantable active device of claim 15, further comprising a power and telemetry coil configured to communicate with a reader and charge the stored energy component.

24. The implantable active device of claim 15, wherein the active channel portion and actuation and sensing module are disposed within a housing.
